(19) United States
(12) Patent Application Publication
(10) Pub. No.: US 2023/0108229 A1
(43) Pub. Date: Apr. 6, 2023

(54) PREDICTION OF INTERFERENCE WITH HOST IMMUNE RESPONSE SYSTEM BASED ON PATHOGEN FEATURES

(71) Applicant: INTERNATIONAL BUSINESS MACHINES CORPORATION, Armonk, NY (US)

(72) Inventors: Akshay Agarwal, Sunnyvale, CA (US); James H. Kaufman, San Jose, CA (US)

(21) Appl. No.: 17/449,022

(22) Filed: Sep. 27, 2021

(51) Int. Cl. G16B 40/20; G16B 20/20; G16B 30/10

(52) U.S. Cl. CPC G16B 40/20; G16B 20/20; G16B 30/10

(57) ABSTRACT

A method, computer system, and a computer program product for pathogen identification is provided. The present invention may include identifying one or more similar pathogens based on a genome of a target pathogen. The present invention may include searching the annotated genome of each of the one or more similar pathogens using a domain code corresponding to a selected condition. The present invention may include identifying one or more epitopes and a protein region of the one or more epitopes for each of the one or more similar pathogens based on the domain code. The present invention may include searching the genome of the target pathogen using an amino acid sequence of the one or more epitopes identified in the annotated genome of the one or more similar pathogens.

### BACKGROUND

The present invention relates generally to the field of computing, and more particularly to identifying pathogen characteristics.

A pathogen may be an organism that causes disease. The different types of pathogens and the severity of the diseases they cause may be diverse. Pathogenic organisms may be of five main types: viruses, bacteria, fungi, protozoa, and worms. Some characteristic features of pathogens may include, but are not limited to including, a mode of transmission, a mechanism of replication, a pathogenesis or means by which the pathogen causes disease, and the response the pathogen elicits. In understanding and/or predicting the characteristics of novel pathogens researchers may utilize related and/or well understood pathogens.

Directly analyzing new pathogens may require domain annotation which can be time consuming and lead to delays in determining likely immune targets and/or effective therapeutics.

### SUMMARY

Embodiments of the present invention disclose a method, computer system, and a computer program product for pathogen identification. The present invention may include identifying one or more similar pathogens based on a genome of a target pathogen. The present invention may include searching the annotated genome of each of the one or more similar pathogens using a domain code corresponding to a selected condition. The present invention may include identifying one or more epitopes and a protein region of the one or more epitopes for each of the one or more similar pathogens based on the domain code. The present invention may include searching the genome of the target pathogen using an amino acid sequence of the one or more epitopes identified in the annotated genome of the one or more similar pathogens.

### BRIEF DESCRIPTION OF THE SEVERAL VIEWS OF THE DRAWINGS

These and other objects, features and advantages of the present invention will become apparent from the following detailed description of illustrative embodiments thereof, which is to be read in connection with the accompanying drawings. The various features of the drawings are not to scale as the illustrations are for clarity in facilitating one skilled in the art in understanding the invention in conjunction with the detailed description. In the drawings:

FIG. 1 illustrates a networked computer environment according to at least one embodiment;

FIG. 2 is an operational flowchart illustrating a process for pathogen identification according to at least one embodiment;

FIG. 3 is a block diagram of internal and external components of computers and servers depicted in FIG. 1 according to at least one embodiment;

FIG. 4 is a block diagram of an illustrative cloud computing environment including the computer system depicted in FIG. 1, in accordance with an embodiment of the present disclosure; and

FIG. 5 is a block diagram of functional layers of the illustrative cloud computing environment of FIG. 4, in accordance with an embodiment of the present disclosure.

### DETAILED DESCRIPTION

Detailed embodiments of the claimed structures and methods are disclosed herein; however, it can be understood that the disclosed embodiments are merely illustrative of the claimed structures and methods that may be embodied in various forms. This invention may, however, be embodied in many different forms and should not be construed as limited to the exemplary embodiments set forth herein. Rather, these exemplary embodiments are provided so that this disclosure will be thorough and complete and will fully convey the scope of this invention to those skilled in the art. In the description, details of well-known features and techniques may be omitted to avoid unnecessarily obscuring the presented embodiments.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user’s computer, partly on the user’s computer, as a stand-alone software package, partly on the user’s computer and partly on a remote computer or entirely on the remote computer or server. In the latter scenario, the remote computer may be connected to the user’s computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The following described exemplary embodiments provide a system, method and program product for pathogen identification. As such, the present embodiment has the capacity to improve the technical field of identifying pathogen characteristics by determining a probability in which a target pathogen causes a selected condition. More specifically, the present invention may include identifying one or more similar pathogens based on a genome of a target pathogen. The present invention may include searching the annotated genome of each of the one or more similar pathogens using a domain code corresponding to a selected condition. The present invention may include identifying one or more epitopes and a protein region of the one or more epitopes for each of the one or more similar pathogens based on the domain code. The present invention may include searching the genome of the target pathogen using an amino acid sequence of the one or more epitopes identified in the annotated genome of the one or more similar pathogens.

As described previously, a pathogen may be an organism that causes disease. The different types of pathogens and the severity of the diseases they cause may be diverse. Pathogenic organisms may be of five main types: viruses, bacteria, fungi, protozoa, and worms. Some characteristic features of pathogens may include, but are not limited to including, a mode of transmission, a mechanism of replication, a pathogenesis or means by which the pathogen causes disease, and the response the pathogen elicits. In understanding and/or predicting the characteristics of novel pathogens researchers may utilize related and/or well understood pathogens.

Directly analyzing new pathogens may require domain annotation which can be time consuming and lead to delays in determining likely immune targets and/or effective therapeutics.

Therefore, it may be advantageous to, among other things, identify one or more similar pathogens based on a genome of a target pathogen, search the annotated genome of each of the one or more similar pathogens using a domain code corresponding to a selected condition, identify one or more epitopes and a protein region of the one or more epitopes for each of the one or more similar pathogens based on the domain code, and search the genome of the target protein using an ammino acid sequence of the one or more epitopes identified in the annotated genome of the one or more similar pathogens.

According to at least one embodiment, the present invention may improve the identification of pathogen characteristics by leveraging mature annotations from other organisms to identify epitopes of the pathogen. These epitopes may indicate specific proteins that may suppress host interferons.

According to at least one embodiment, the present invention may improve the identification of conditions caused by novel pathogens by utilizing laboratory confirmed reference epitopes to search a genome of the novel pathogen.

According to at least one embodiment, the present invention may improve the ability to understand pathogens prior to domain annotation and/or functional annotation by using known conditions caused by one or more similar pathogens to search the genome of the novel pathogen for epitopes derived from the known conditions of the one or more similar pathogens.

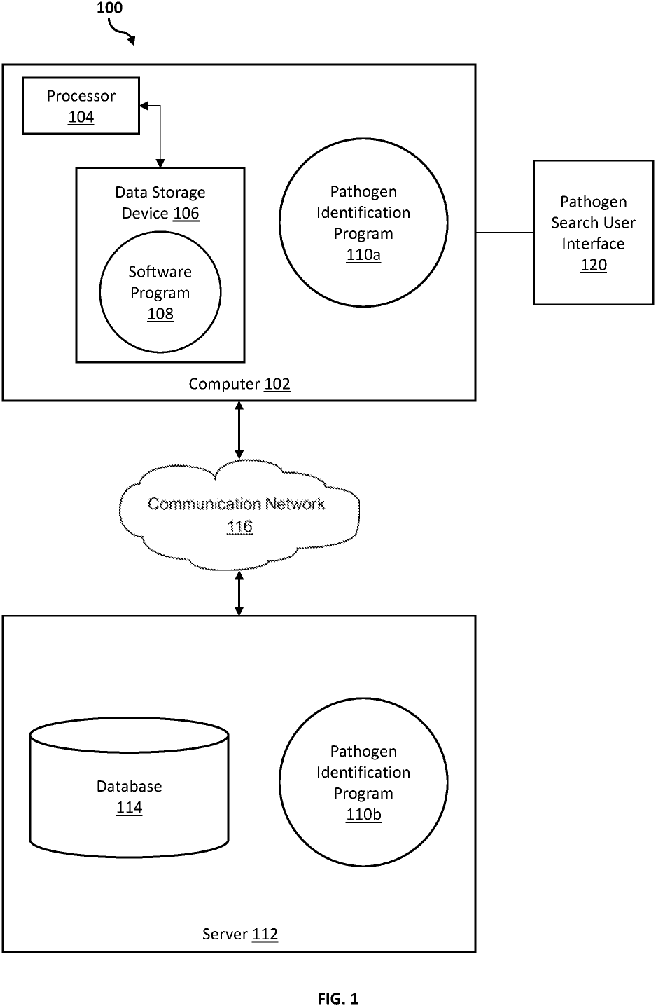

Referring to FIG. 1, an exemplary networked computer environment 100 in accordance with one embodiment is depicted. The networked computer environment 100 may include a computer 102 with a processor 104 and a data storage device 106 that is enabled to run a software program 108 and a pathogen identification program 110a. The networked computer environment 100 may also include a server 112 that is enabled to run a pathogen identification program 110b that may interact with a database 114 and a communication network 116. The networked computer environment 100 may include a plurality of computers 102 and servers 112, only one of which is shown. The networked computer environment 100 may include a pathogen search user interface. The communication network 116 may include various types of communication networks, such as a wide area network (WAN), local area network (LAN), a telecommunication network, a wireless network, a public switched network and/or a satellite network. It should be appreciated that FIG. 1 provides only an illustration of one implementation and does not imply any limitations with regard to the environments in which different embodiments may be implemented. Many modifications to the depicted environments may be made based on design and implementation requirements.

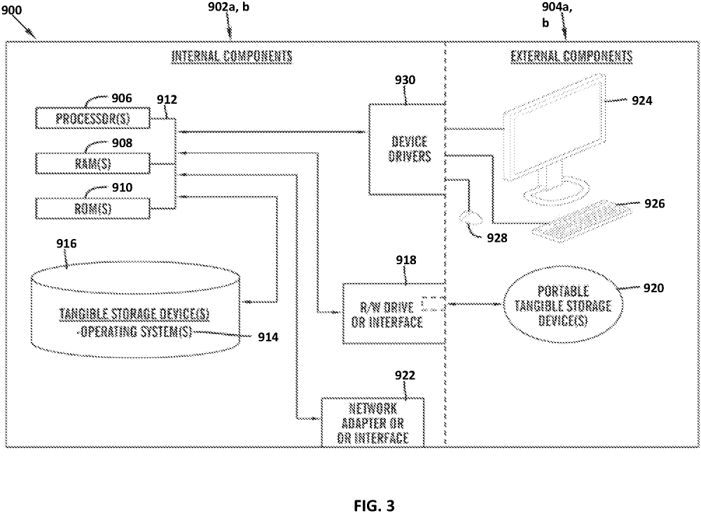

The client computer 102 may communicate with the server computer 112 via the communications network 116. The communications network 116 may include connections, such as wire, wireless communication links, or fiber optic cables. As will be discussed with reference to FIG. 3, server computer 112 may include internal components 902a and external components 904a, respectively, and client computer 102 may include internal components 902b and external components 904b, respectively. Server computer 112 may also operate in a cloud computing service model, such as Software as a Service (SaaS), Platform as a Service (PaaS), or Infrastructure as a Service (IaaS). Server 112 may also be located in a cloud computing deployment model, such as a private cloud, community cloud, public cloud, or hybrid cloud. Client computer 102 may be, for example, a mobile device, a telephone, a personal digital assistant, a netbook, a laptop computer, a tablet computer, a desktop computer, or any type of computing devices capable of running a program, accessing a network, and accessing a database 114. According to various implementations of the present embodiment, the pathogen identification program 110a, 110b may interact with a database 114 that may be embedded in various storage devices, such as, but not limited to a computer/mobile device 102, a networked server 112, or a cloud storage service.

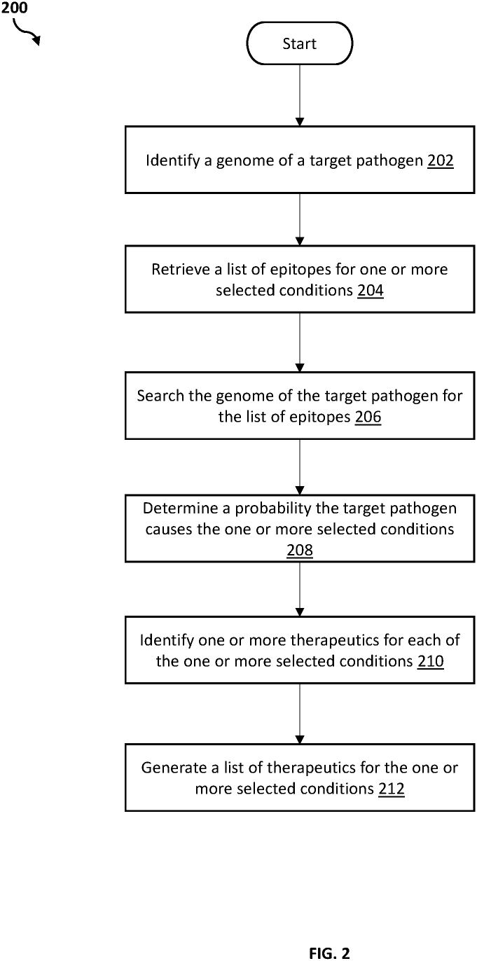

According to the present embodiment, a user using a client computer 102 or a server computer 112 may use the pathogen identification program 110a, 110b (respectively) to determine a probability in which a target pathogen causes a selected condition. The pathogen identification method is explained in more detail below with respect to FIG. 2.

Referring now to FIG. 2, an operational flowchart illustrating the exemplary pathogen identification process 200 used by the pathogen identification program 110a and 110b (hereinafter pathogen identification program 110) according to at least one embodiment is depicted.

At 202, the pathogen identification program 110 identifies a genome of a target pathogen. The genome of the target pathogen may be a complete set of either RNA (Ribonucleic acid) or DNA (Deoxyribonucleic acid). The genome of the target pathogen may be comprised of nucleotide sequences and the nucleotide sequences may include both coding regions and/or noncoding regions. Coding regions may encode for proteins and noncoding regions may not encode for proteins. The genome of the target pathogen may not be annotated and/or partially annotated, such that domain annotations for protein regions (e.g., domain annotations for protein encoding regions of the genome) may not be mature and/or available for the genome of the target pathogen. Accordingly, the genome of the pathogen may not include domain codes (e.g., functional codes). Domain codes (e.g., functional codes) may be utilized in denoting a function of a particular protein region of the genome.

The pathogen identification program 110 may identify the genome of the target pathogen in a knowledge corpus (e.g., database 114) maintained by the pathogen identification program. In an embodiment, the pathogen identification program 110 may identify the genome of the target pathogen in the knowledge corpus (e.g., database 114) using a machine learning classification model, such as logistic regression, naive Bayes, support vector machines, artificial neural networks, random forests, amongst other algorithms. In an embodiment, an ensemble learning technique may be employed that uses multiple machine leaning algorithms together to assure better identification when compared with the identification of a single machine learning model. The knowledge corpus (e.g., database 114) may maintain the genomes for a plurality of organisms. Alternatively, the pathogen identification program 110 may retrieve the genome of the target pathogen from one or more publicly available resources and/or external databases. The genome of the target pathogen identified by the pathogen identification program 110 may be confirmed by a user. In another embodiment, the genome of the target pathogen may also be manually identified and uploaded to the pathogen identification program 110 by a user in a pathogen search user interface 120. The target pathogen in which the pathogen identification program 110 identifies the genome may be manually selected by a user of the pathogen identification program 110.

The pathogen identification program 110 may utilize the genome of the target pathogen in identifying one or more similar pathogens. The one or more similar pathogens may be identified utilizing one or more identification methods, such as, but not limited to, homology-based methods, ab initio methods, amongst other identification methods. The similar pathogens may also be identified using a sequence-based genomic database, wherein the similar pathogens are identified using the genome of the pathogen. The sequence-based genomic database may be maintained by the pathogen identification program 110 within the knowledge corpus (e.g., database 114) and/or accessed by the pathogen identification program 110 from one or more publicly available resources and/or external databases. The similar pathogens may also be manually identified by a user based on at least characteristics such as size, shape, content (e.g., RNA or DNA, enveloped or not), taxonomic classification, amongst other pathogen characteristics. The pathogen identification program 110 may also use one or more taxonomic classification tools and/or methods in identifying the one or more similar pathogens. The genome of each of the similar pathogens may be at least partially annotated.

The pathogen identification program 110 may utilize the one or more similar pathogens in identifying a plurality of conditions. The plurality of conditions may be medical conditions, side effects, symptoms, interference with biological pathways, ability to bind with certain proteins, and/or other conditions linked to and/or caused by the one or more similar pathogens. Each of the plurality of conditions may each have a corresponding domain code (e.g., corresponding functional code) such as an InterPRO Bioscience® codes (InterPRO Bioscience® codes and all InterPRO Bioscience-based trademarks are trademarks or registered trademarks of InterPRO Bioscience Incorporated in the United States, and/or other countries), amongst other codes utilized in the genomic industry. The corresponding domain code (e.g., corresponding functional code) may correspond to viral genes of the similar pathogen’s genome, these viral genes may encode for proteins called virulence factors and these virulence factors contribute to the similar pathogen’s ability to cause a condition.

The pathogen identification program 110 may retrieve one or more annotated genomes for each of the one or more similar pathogens identified. The pathogen identification program 110 may retrieve more than one genome for a similar pathogen if the genome of the similar pathogen has more than one variation. The annotated genomes for each of the one or more similar pathogens may be retrieved from the knowledge corpus (e.g., database 114) and/or from one or more publicly available resources and/or external databases. The pathogen identification program 110 may utilize the corresponding domain codes (e.g., functional codes) to search the annotated genomes of each of the one or more similar pathogens. The pathogen identification program 110 may identify a protein region of the annotated genome based on a search of the annotated genome utilizing the corresponding domain code. The protein region may be a protein sequence of the annotated genome in which the corresponding domain code may be identified.

The pathogen identification program 110 may utilize the protein sequence of the protein region in identifying one or more epitopes within the protein region. An epitope may be a sub-string and/or multiple sub-strings of amino acids within a protein region which may be recognized by a host immune system. The sub-string and/or multiple sub-strings may be continuous and/or discontinuous. The epitope may be the amino acid segment of the protein region in which antibodies of the host immune system bind. Amino acids may be the monomers that make up proteins. Each amino acid may include a similar fundamental structure, which may be comprised of a central carbon atom bonded to an amino group, a carboxyl group, a hydrogen atom, and a variable atom or group of atoms bonded to the central carbon atom. Protein molecules may be comprised of a string of amino acids in a particular order (e.g., amino acid sequence). All proteins may be constructed from one of 20 amino acids. Accordingly, epitopes may determine an immune response and each of the protein sequences may include one or more epitopes. The one or more epitopes may be laboratory confirmed epitopes (e.g., known epitope amino acid sequences). The pathogen identification program 110 may store an epitope list for each condition in which it was derived.

For example, the pathogen identification program 110 may identify five medical conditions associated with the one or more similar pathogens, Condition 1, Condition 2, Condition 3, Condition 4, Condition 5. Each of the five medical conditions has a corresponding domain code, Condition 1 may be IPR00001, Condition 2 may be IPR00002, Condition 3 may be IPR00003, Condition 4 may be IPR00004, and Condition 5 may be IPR00005. The pathogen identification program 110 may utilize the corresponding domain code to search the annotated genome of each of the one or more similar pathogens and identify the protein sequence of the one or more similar pathogens associated with the condition. The pathogen identification program 110 may identify 7 epitopes within the protein sequence identified by searching IPR00001, 4 epitopes within the protein sequence identified by searching IPR00002, 2 epitopes within the protein sequence identified by searching IPR00003, 3 epitopes within the protein sequence identified by searching IPR00004, and 5 epitopes within the protein sequence identified by searching IPR00005. Accordingly, the pathogen identification program 110 may store 7 epitopes for Condition 1, 4 epitopes for Condition 2, 2 epitopes for Condition 3, 3 epitopes for Condition 4, and/or 5 epitopes for Condition 5. The pathogen identification program 110 may store each epitope amino acid sequence and/or the condition in which it was derived in the knowledge corpus (e.g., database 114).

In another embodiment, as will be explained in more detail below with respect to step 210, the genome of the target pathogen may be annotated such that the genome of the protein regions include mature domain annotations. The pathogen identification program 110 may continuously monitor one or more publicly available resources and/or external databases for annotation updates of the target pathogen genome and/or manually receive the updated annotations for the target pathogen genome from a user in the pathogen search user interface 120.

At 204, the pathogen identification program 110 retrieves a list of epitopes for one or more selected conditions. The pathogen identification program 110 may retrieve the list of epitopes for the one or more selected conditions from the knowledge corpus (e.g., database 114). The one or more conditions may be selected by a user of the pathogen identification program 110 using the pathogen search user interface 120. The conditions may be selected from a list of conditions identified for the similar pathogens. The list of conditions may be displayed to the user in the pathogen search user interface. The pathogen search user interface 120 may be displayed by the pathogen identification program 110 in at least an internet browser, dedicated software application, or as an integration with a third party software application.

The pathogen identification program 110 may retrieve the list of epitopes for the selected conditions to search the genome of the target pathogen identified at step 202. The plurality of conditions may be medical conditions, side effects, symptoms, interference with biological pathways, ability to bind with certain proteins, and/or other conditions linked to and/or caused by the one or more similar pathogens. Continuing with the above example, Condition 1 may be nausea and Condition 2 may be elevated heart rate, the user may select nausea and elevated heart rate and the pathogen identification program 110 may retrieve the list of 7 epitopes for Condition 1 and the list of 4 epitopes for Condition 2 from the knowledge corpus (e.g., database 114). In another example, Condition 1 may be interference with Biological Pathway A, Condition 2 may be interference with Biological Pathway B, and Condition 3 may be ability to bind with Protein 1. The user may select interference with Biological Pathway A, interference with Biological Pathway B, and ability to bind with Protein 1 in the pathogen search user interface and the pathogen identification program 110 may retrieve the list of 7 epitopes for Condition 1, the list of 4 epitopes for Condition 2, and the list of 2 epitopes for Condition 3 from the knowledge corpus (e.g., database 114).

At 206, the pathogen identification program 110 searches the genome of the target pathogen for the list of epitopes of the selected conditions. The pathogen identification program 110 may utilize the amino acid sequence of each epitope in searching the genome of the target pathogen. The pathogen identification program 110 may search subsections of the target pathogen genome using the protein regions of the epitopes identified in the similar pathogens.

The pathogen identification program 110 may utilize one or more search methods in searching the genome of the target pathogen. The search methods may include but are not limited to including, exact string matching and approximate string matching (e.g., fuzzy string search). The search methods utilized by the pathogen identification program 110 may be utilized in ranking epitope-domain matches. As will be explained in more detail below, the ranking of the epitope-domain matches may be utilized in determining a probability score the target pathogen may cause a condition. The epitope-domain match may be a sequence of amino acids found within the genome of the target pathogen. The epitope-domain match may be part of a protein region of the genome.

The pathogen identification program 110 may rank the exact matches the highest. The exact matches may be ranked according to the number of amino acids in the exact string match sequence. The pathogen identification program 110 may rank the non-exact matches above a pre-defined threshold using a percentage of approximate string matches. For example, the pathogen identification program 110 may identify two exact epitope-domain matches. One of the epitope-domain matches may be an exact match of 6 amino acids and the other epitope-domain match may be an exact match of 9 amino acids. The pathogen identification program 110 may rank the epitope-domain match of 9 amino acids above the epitope-domain match of 6 amino acids. The pathogen identification program 110 may also identify five non-exact epitope-domain matches. The first non-exact epitope-domain match may match 8 of 9 amino acids, the second non-exact epitope genome match may match 6 of 7 amino acids, the third non-exact epitope genome match may match 5 of 8 amino acids, the fourth non-exact epitope genome match may match 7 of 8 amino acids, and the fifth non-exact epitope genome match may match 6 of 8 amino acids. The pre-defined threshold may be an 80% amino acid match. In this example, the pathogen identification program 110 would only rank the first, second, and fourth non-exact epitope-domain matches.

In other embodiments, the pathogen identification program 110 may utilize one or more machine learning models and/or one or more statistical analysis models in identifying epitope-domain matches within the genome of the target pathogen. The one or more machine learning models may include at least affinity determining models and/or affinity predicting models. The one or more machine learning models may be utilized by the pathogen identification program 110 in performing a clustering analysis of the genome of the target pathogen in identifying epitope-domain matches. The one or more statistical models may include statistical models such as Basic Local Alignment Search Tool® (BLAST® and all BLAST®-based trademarks are trademarks or registered trademarks of the National Library of Medicine in the United States, and/or other countries). The pathogen identification program 110 may utilize an expect value (e.g., Expect Value) in identifying the one or more epitope-domain matches using statistical models. In this embodiment, the epitope-domain matches may be independent of amino acid length.

At 208, the pathogen identification program 110 determines a probability in which the target pathogen causes each of the selected conditions. The pathogen identification program 110 may determine the probability in which the target pathogen causes each of the selected conditions based on at least the ranking of the epitope-domain matches and/or the protein region of the epitope-domain match. The pathogen identification program 110 may generate a probability score for each of the selected conditions. The probability score may represent a probability the target pathogen may cause each selected condition.

The pathogen identification program 110 may compare the protein region in which the epitope-domain match was identified in the genome of the target pathogen with the protein region in which the epitope was identified in the similar pathogen. If the protein region of the target pathogen genome and the protein region of the similar pathogen genome match the pathogen identification program 110 may weight the probability score higher for the selected condition than if the protein region for the target pathogen and similar pathogen did not match. The pathogen identification program 110 may determine whether the target pathogen may cause each of the selected conditions based on the probability score. The pathogen identification program 110 may compare the probability score for each of the selected conditions to a threshold score, if the probability score exceeds the threshold score for the selected condition the pathogen identification program 110 may determine the target pathogen may cause the selected condition.

The threshold score may be pre-determined by a user within the pathogen search user interface 120 and/or determined by the pathogen identification program 110 based on at least completeness of genome annotation for the similar pathogens. The threshold score may be intermittently adjusted based on the completeness of the genome annotation for the similar pathogens and/or as the target pathogen genome may be annotated.

At 210, the pathogen identification program 110 identifies one or more therapeutics for each of the selected conditions in which the probability score that the target pathogen causes the selected condition exceeds the threshold score. The pathogen identification program 110 may identify the one or more therapeutics for each of the one or more selected conditions using at least therapeutics utilized in treatment of the similar pathogens. The one or more therapeutics may block and/or inhibit the domain of the epitope-domain matches of the target pathogen.

The pathogen identification program 110 may search the genome of the similar pathogens using the epitope-domain matches of the target pathogen. The pathogen identification program 110 may utilize a domain of the protein region of the epitope in identifying the therapeutics. The domain may be a specific combination of secondary structures organized into a characteristic three-dimensional structure. Protein domains may correspond to structural domains which may be self-stabilizing and fold independently of the rest of the protein sequence. Protein domains may occur independently and/or as part of a complex multidomain protein architecture which my evolve by at least domain accretion, domain loss, and/or domain recombination.

In another embodiment, the target pathogen may have an annotated and/or partially annotated genome. In this embodiment, the pathogen identification program 110 may generate a list of epitopes and the domain (e.g., protein region) in which they are identified. In this embodiment, the pathogen identification program 110 may utilize the genomes of both similar and/or dissimilar pathogens. The pathogen identification program 110 may search the similar and/or dissimilar pathogen genomes utilizing the list of epitopes generated for the target pathogen and may compare the domain (e.g., protein region) in which the epitope may be identified within the similar and/or dissimilar pathogen genomes to the domain (e.g., protein region) of the target pathogen. The pathogen identification program 110, may utilize at least the search methods, one or more machine learning models, and/or statistical models outlined above with respect to step 206 in searching the similar and/or dissimilar pathogen genomes. In this embodiment, the pathogen identification program 110 may generate a list of therapeutics that block and/or inhibit the domain of the epitope-domain matches. In another embodiment, the pathogen identification program 110 may identify one or more pathways of the domain for the epitope-domain matches and utilize one or more publicly available resources and/or databases to determine one or more therapeutics which may block and/or inhibit the one or more pathways of the domain.

In an embodiment, the pathogen identification program 110 may continuously monitor one or more publicly available resources and/or external databased using at least the search methods, machine learning models, and/or statistical models outlined above. The pathogen identification program 110 may also receive annotation updates of the target pathogen genome from the user in the pathogen search user interface 120. The pathogen identification program 110 continuously update the list of epitope-domain matches based on a most recent genome of the target pathogen. For example, the pathogen identification program 110 may not have identified an epitope-domain match for an original genome of the target pathogen but identified the epitope-domain for the most recent genome of the target pathogen. In this example, the pathogen identification program 110 may determine an increased virulence for the target pathogen based on the increased number of epitopes identified for a condition.

At 212, the pathogen identification program 110 generates a list of therapeutics for the each of the selected conditions in which the probability score of the target pathogen causing the selected condition exceeds the threshold score. Each therapeutic of the list of may utilize a blocking function for the domain of the protein region in which the epitope may be identified. The list of therapeutics may be ranked according to a similarity score, the similarity score may be determined for each epitope-domain tuple. The pathogen identification program 110 may calculate an edit distance between each epitope-domain tuple based on an overlap of the epitope amino acid sequence and the domain protein region. The list of therapeutics may be displayed to the user in the pathogen search user interface 120.

It may be appreciated that FIG. 2 provides only an illustration of one embodiment and do not imply any limitations with regard to how different embodiments may be implemented. Many modifications to the depicted embodiment(s) may be made based on design and implementation requirements.

FIG. 3 is a block diagram 900 of internal and external components of computers depicted in FIG. 1 in accordance with an illustrative embodiment of the present invention. It should be appreciated that FIG. 3 provides only an illustration of one implementation and does not imply any limitations with regard to the environments in which different embodiments may be implemented. Many modifications to the depicted environments may be made based on design and implementation requirements.

Data processing system 902, 904 is representative of any electronic device capable of executing machine-readable program instructions. Data processing system 902, 904 may be representative of a smart phone, a computer system, PDA, or other electronic devices. Examples of computing systems, environments, and/or configurations that may represented by data processing system 902, 904 include, but are not limited to, personal computer systems, server computer systems, thin clients, thick clients, hand-held or laptop devices, multiprocessor systems, microprocessor-based systems, network PCs, minicomputer systems, and distributed cloud computing environments that include any of the above systems or devices.

User client computer 102 and network server 112 may include respective sets of internal components 902 a, b and external components 904 a, b illustrated in FIG. 3. Each of the sets of internal components 902 a, b includes one or more processors 906, one or more computer-readable RAMs 908 and one or more computer-readable ROMs 910 on one or more buses 912, and one or more operating systems 914 and one or more computer-readable tangible storage devices 916. The one or more operating systems 914, the software program 108, and the pathogen identification program 110a in client computer 102, and the pathogen identification program 110b in network server 112, may be stored on one or more computer-readable tangible storage devices 916 for execution by one or more processors 906 via one or more RAMs 908 (which typically include cache memory). In the embodiment illustrated in FIG. 3, each of the computer-readable tangible storage devices 916 is a magnetic disk storage device of an internal hard drive. Alternatively, each of the computer-readable tangible storage devices 916 is a semiconductor storage device such as ROM 910, EPROM, flash memory or any other computer-readable tangible storage device that can store a computer program and digital information.

Each set of internal components 902 a, b also includes a R/W drive or interface 918 to read from and write to one or more portable computer-readable tangible storage devices 920 such as a CD-ROM, DVD, memory stick, magnetic tape, magnetic disk, optical disk or semiconductor storage device. A software program, such as the software program 108 and the pathogen identification program 110a and 110b can be stored on one or more of the respective portable computer-readable tangible storage devices 920, read via the respective R/W drive or interface 918 and loaded into the respective hard drive 916.

Each set of internal components 902 a, b may also include network adapters (or switch port cards) or interfaces 922 such as a TCP/IP adapter cards, wireless wi-fi interface cards, or 3G or 4G wireless interface cards or other wired or wireless communication links. The software program 108 and the pathogen identification program 110a in client computer 102 and the pathogen identification program 110b in network server computer 112 can be downloaded from an external computer (e.g., server) via a network (for example, the Internet, a local area network or other, wide area network) and respective network adapters or interfaces 922. From the network adapters (or switch port adaptors) or interfaces 922, the software program 108 and the pathogen identification program 110a in client computer 102 and the pathogen identification program 110b in network server computer 112 are loaded into the respective hard drive 916. The network may comprise copper wires, optical fibers, wireless transmission, routers, firewalls, switches, gateway computers and/or edge servers.

Each of the sets of external components 904 a, b can include a computer display monitor 924, a keyboard 926, and a computer mouse 928. External components 904 a, b can also include touch screens, virtual keyboards, touch pads, pointing devices, and other human interface devices. Each of the sets of internal components 902 a, b also includes device drivers 930 to interface to computer display monitor 924, keyboard 926 and computer mouse 928. The device drivers 930, R/W drive or interface 918 and network adapter or interface 922 comprise hardware and software (stored in storage device 916 and/or ROM 910).

It is understood in advance that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g. networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

### Characteristics Are as Follows

On-demand self-service: a cloud consumer can unilaterally provision computing capabilities, such as server time and network storage, as needed automatically without requiring human interaction with the service’s provider.

Broad network access: capabilities are available over a network and accessed through standard mechanisms that promote use by heterogeneous thin or thick client platforms (e.g., mobile phones, laptops, and PDAs).

Resource pooling: the provider’s computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported providing transparency for both the provider and consumer of the utilized service.

### Service Models Are as Follows

Software as a Service (SaaS): the capability provided to the consumer is to use the provider’s applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

### Deployment Models Are as Follows

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure comprising a network of interconnected nodes.

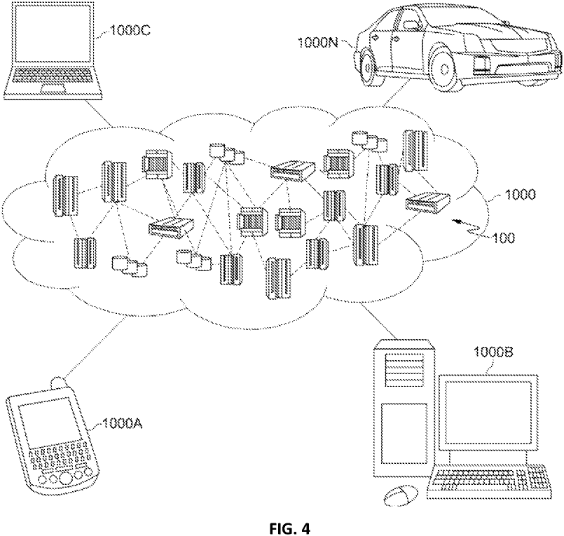

Referring now to FIG. 4, illustrative cloud computing environment 1000 is depicted. As shown, cloud computing environment 1000 comprises one or more cloud computing nodes 100 with which local computing devices used by cloud consumers, such as, for example, personal digital assistant (PDA) or cellular telephone 1000A, desktop computer 1000B, laptop computer 1000C, and/or automobile computer system 1000N may communicate. Nodes 100 may communicate with one another. They may be grouped (not shown) physically or virtually, in one or more networks, such as Private, Community, Public, or Hybrid clouds as described hereinabove, or a combination thereof. This allows cloud computing environment 1000 to offer infrastructure, platforms and/or software as services for which a cloud consumer does not need to maintain resources on a local computing device. It is understood that the types of computing devices 1000A-N shown in FIG. 4 are intended to be illustrative only and that computing nodes 100 and cloud computing environment 1000 can communicate with any type of computerized device over any type of network and/or network addressable connection (e.g., using a web browser).

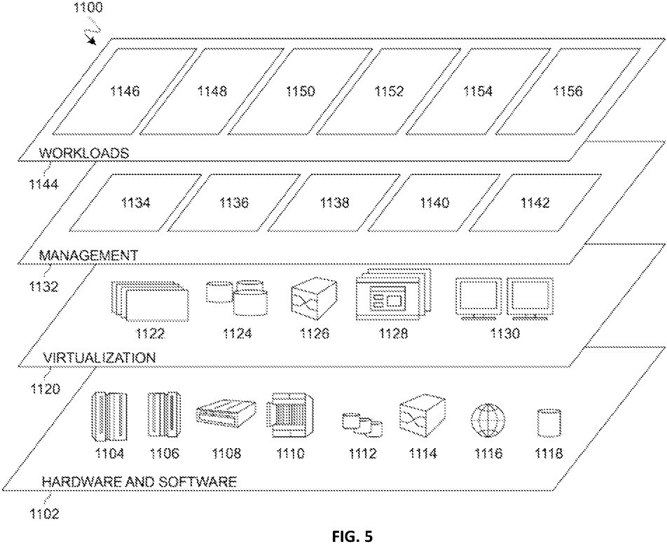

Referring now to FIG. 5, a set of functional abstraction layers 1100 provided by cloud computing environment 1000 is shown. It should be understood in advance that the components, layers, and functions shown in FIG. 5 are intended to be illustrative only and embodiments of the invention are not limited thereto. As depicted, the following layers and corresponding functions are provided:

Hardware and software layer 1102 includes hardware and software components. Examples of hardware components include: mainframes 1104; RISC (Reduced Instruction Set Computer) architecture based servers 1106; servers 1108; blade servers 1110; storage devices 1112; and networks and networking components 1114. In some embodiments, software components include network application server software 1116 and database software 1118.

Virtualization layer 1120 provides an abstraction layer from which the following examples of virtual entities may be provided: virtual servers 1122; virtual storage 1124; virtual networks 1126, including virtual private networks; virtual applications and operating systems 1128; and virtual clients 1130.

In one example, management layer 1132 may provide the functions described below. Resource provisioning 1134 provides dynamic procurement of computing resources and other resources that are utilized to perform tasks within the cloud computing environment. Metering and Pricing 1136 provide cost tracking as resources are utilized within the cloud computing environment, and billing or invoicing for consumption of these resources. In one example, these resources may comprise application software licenses. Security provides identity verification for cloud consumers and tasks, as well as protection for data and other resources. User portal 1138 provides access to the cloud computing environment for consumers and system administrators. Service level management 1140 provides cloud computing resource allocation and management such that required service levels are met. Service Level Agreement (SLA) planning and fulfillment 1142 provide pre-arrangement for, and procurement of, cloud computing resources for which a future requirement is anticipated in accordance with an SLA.

Workloads layer 1144 provides examples of functionality for which the cloud computing environment may be utilized. Examples of workloads and functions which may be provided from this layer include: mapping and navigation 1146; software development and lifecycle management 1148; virtual classroom education delivery 1150; data analytics processing 1152; transaction processing 1154; and pathogen identification 1156. A pathogen identification program 110a, 110b provides a way to determine a probability in which a target pathogen causes a selected condition.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope of the described embodiments. The terminology used herein was chosen to best explain the principles of the embodiments, the practical application or technical improvement over technologies found in the marketplace, or to enable others of ordinary skill in the art to understand the embodiments disclosed herein.

The present disclosure shall not be construed as to violate or encourage the violation of any local, state, federal, or international law with respect to privacy protection.

What is claimed is:

1. A method for pathogen identification, the method comprising:
identifying one or more similar pathogens based on a genome of a target pathogen, wherein each of the one or more similar pathogens have an annotated genome;
searching the annotated genome of each of the one or more similar pathogens using a domain code corresponding to a selected condition;
identifying one or more epitopes and a protein region of the one or more epitopes for each of the one or more similar pathogens based on the domain code; and
searching the genome of the target pathogen using an amino acid sequence of the one or more epitopes identified in the annotated genome of the one or more similar pathogens.

2. The method of claim 1, wherein searching the genome of the target pathogen utilizing the amino acid sequence of the one or more epitopes further comprises:
searching the genome of the target pathogen using one or more search methods; and
ranking one or more epitope-domain matches identified in the genome of the target pathogen.

3. The method of claim 2, wherein the one or more search methods include at least exact string matching and approximate string matching.

4. The method of claim 2, further comprising:
determining a probability in which the target pathogen causes a selected condition, wherein the probability is represented using a probability score and the probability score is determined based on at least an epitope-domain match and a protein region of the epitope-domain match.

5. The method of claim 4, further comprising:
identifying one or more therapeutics for the selected condition if the probability score exceeds a threshold score.

6. The method of claim 5, wherein the one or more therapeutics are ranked according to a similarity score.

7. The method of claim 1, wherein the selected condition is selected by a user using a pathogen search user interface.

8. A computer system for pathogen identification, comprising:
one or more processors, one or more computer-readable memories, one or more computer-readable tangible storage medium, and program instructions stored on at least one of the one or more tangible storage medium for execution by at least one of the one or more processors via at least one of the one or more memories, wherein the computer system is capable of performing a method comprising:
identifying one or more similar pathogens based on a genome of a target pathogen, wherein each of the one or more similar pathogens have an annotated genome;
searching the annotated genome of each of the one or more similar pathogens using a domain code corresponding to a selected condition;
identifying one or more epitopes and a protein region of the one or more epitopes for each of the one or more similar pathogens based on the domain code; and
searching the genome of the target pathogen using an amino acid sequence of the one or more epitopes identified in the annotated genome of the one or more similar pathogens.

9. The computer system of claim 8, wherein searching the genome of the target pathogen utilizing the amino acid sequence of the one or more epitopes further comprises:
searching the genome of the target pathogen using one or more search methods; and
ranking one or more epitope-domain matches identified in the genome of the target pathogen.

10. The computer system of claim 9, wherein the one or more search methods include at least exact string matching and approximate string matching.

11. The computer system of claim 9, further comprising:
determining a probability in which the target pathogen causes a selected condition, wherein the probability is represented using a probability score and the probability score is determined based on at least an epitope-domain match and a protein region of the epitope-domain match.

12. The computer system of claim 11, further comprising:
identifying one or more therapeutics for the selected condition if the probability score exceeds a threshold score.

13. The computer system of claim 12, wherein the one or more therapeutics are ranked according to a similarity score.

14. The computer system of claim 8, wherein the selected condition is selected by a user using a pathogen search user interface.

15. A computer program product for pathogen identification, comprising:
one or more non-transitory computer-readable storage media and program instructions stored on at least one of the one or more tangible storage media, the program instructions executable by a processor to cause the processor to perform a method comprising:
identifying one or more similar pathogens based on a genome of a target pathogen, wherein each of the one or more similar pathogens have an annotated genome;
searching the annotated genome of each of the one or more similar pathogens using a domain code corresponding to a selected condition;
identifying one or more epitopes and a protein region of the one or more epitopes for each of the one or more similar pathogens based on the domain code; and
searching the genome of the target pathogen using an amino acid sequence of the one or more epitopes identified in the annotated genome of the one or more similar pathogens.

16. The computer program product of claim 15, wherein searching the genome of the target pathogen utilizing the amino acid sequence of the one or more epitopes further comprises:
searching the genome of the target pathogen using one or more search methods; and
ranking one or more epitope-domain matches identified in the genome of the target pathogen.

17. The computer program product of claim 16, wherein the one or more search methods include at least exact string matching and approximate string matching.

18. The computer program product of claim 16, further comprising:
determining a probability in which the target pathogen causes a selected condition, wherein the probability is represented using a probability score and the probability score is determined based on at least an epitope-domain match and a protein region of the epitope-domain match.

19. The computer program product of claim 18, further comprising:
identifying one or more therapeutics for the selected condition if the probability score exceeds a threshold score.

20. The computer program product of claim 19, wherein the one or more therapeutics are ranked according to a similarity score.
